(19) United States
(12) Patent Application Publication
(10) Pub. No.: US 2012/0202849 A1
(43) Pub. Date: Aug. 9, 2012

(54) PHARMACEUTICAL COMPOSITIONS FOR THE TREATMENT/PROPHYLAXIS OF NON-ALCOHOLIC FATTY LIVER DISEASE

(76) Inventor: Anil Pareek, Mumbai (IN)

(21) Appl. No.: 13/501,342

(22) PCT Filed: Oct. 12, 2010
(86) PCT No.: PCT/IN2010/000677

§ 371 Date: Apr. 11, 2012

(30) Foreign Application Priority Data
Oct. 12, 2009 (IN) 2375/MUM/2009

(51) Int. Cl. A61K 31/4706; A61P 1/16

(52) U.S. Cl. 514/313; 546/312

(57) ABSTRACT

Disclosed herein is a novel synergistic pharmaceutical composition comprising hydroxychloroquine with insulin sensitizing agents and lipid lowering agents such as statins along with pharmaceutical excipients/carriers useful in treating Non-Alcoholic Fatty Liver Disease.

### TECHNICAL FIELD OF INVENTION

The present invention relates to novel synergistic pharmaceutical compositions comprising combinations of hydroxychloroquine or its pharmaceutically acceptable salts and at least one lipid lowering agent and at least one insulin sensitizing agent; to pharmaceutical kits containing such combinations; methods of using such combinations to treat subjects suffering from a Non-Alcoholic Fatty Liver Disease (NAFLD); and to treat subjects susceptible to develop with symptoms of Non-Alcoholic Fatty Liver Disease or prophylaxis of NAFLD, including humans.

### BACKGROUND OF THE INVENTION

Hydroxychloroquine, disclosed in U.S. Pat. No. 2,546,658 is a disease modifying antirheumatic drug (DMARD) and is being used in rheumatology for past four decades. The use of hydroxychloroquine is well established in rheumatoid arthritis and systemic lupus erythematosus.

Statins are compounds that inhibit HMGCoA reductase. HMGCoA reductase catalyzes the conversion of 3-hydroxy-O-methylglutaryl-coenzyme A (HMGCoA) to mevalonate, which is an early and rate-limiting step in the cholesterol biosynthetic pathway and because of inhibiting the HMGCoA reductase enzyme, statins are acting as potent lipid lowering agents. The compounds in pharmaceutical use from Statins class include simvastatin or its salts (disclosed in U.S. Pat. No. 4,444,784); pravastatin or its salts (disclosed in U.S. Pat. No. 4,346,227); mevastatin or its salts (disclosed in U.S. Pat. No. 3,983,140); fluvastatin or its salts (disclosed in U.S. Pat. No. 4,739,073); lovastatin (disclosed in U.S. Pat. No. 4,231,938); Atorvastatin or its salts (disclosed in U.S. Pat. No. 4,681,893) and Rosuvastatin (disclosed in U.S. Pat. No. RE37314).

Biguanide compounds, especially metformin (disclosed in U.S. Pat. No. 3,174,901), are known to reduce hyperinsulinaemia and improves hepatic insulin resistance. Its major site of action appears to be in the mitochondria, and it has been shown to stimulate pyruvate-kinase, fatty acid beta-oxidation, anaerobic respiration (i.e. lactate production) as well as suppress the expression of lipogenic enzymes.

Non-alcoholic fatty liver disease (NAFLD) comprises a spectrum of liver disease characterized from simple fatty liver changes (macrovascular fatty change) to non-alcoholic steatohepatitis (NASH), and cirrhosis that is not related to consumption of alcohol in amounts considered detrimental to the liver. (Adv Ant Pathol 2002; 9(1):37-51).

NAFLD is now recognized as the most common cause of cryptogenic cirrhosis. (JAMA 2003; 289 (22):3000-4). NAFLD affects 10 to 24% of general population in various countries. The prevalence of NAFLD ranges from 57.5% to 74% in obese persons. NAFLD affects 2.6% of children and 22.5% to 52.8% of obese children. (Dig Dis Sci 1995; 40 2002-9).

NAFLD begins with fatty liver, progressing through NASH, and ending with cirrhosis. Fatty liver (steatosis) is characterized by accumulation of fat in liver cells without inflammation or scarring. (Pak J Med Sci 2005; 21(4):472-475). Only fraction of patients with simple fatty liver will develop NASH, which involves steatosis, inflammation (hepatitis), and scarring (fibrosis) in the liver. NASH can ultimately lead to scarring of the liver (fibrosis) and then to irreversible advanced scarring (cirrhosis). Cirrhosis is the last and the most severe stage in the NAFLD spectrum (J Medicine 2007; 8: 17-27). The cause of NAFLD is multifactorial; however, the most common risk factor is the presence of the metabolic syndrome that includes insulin resistance, diabetes mellitus, obesity, dyslipidemia (mainly hypertriglyceridemia), and hypertension. (Hypertension. 2005; 45: 1012-1018).

There are currently no approved therapeutic regimens for treatment of NAFLD. Metformin was found to reverse the hepatomegaly and steatosis in previous studies. In a phase II clinical trial of metformin as a treatment for non-diabetic paediatric non alcoholic steatohepatitis, metformin shows improvements in the liver chemistry, liver fat, insulin sensitivity and quality of life (Aliment Pharmacol Therap. 2005; 21 (7):871-9). Metformin treatment has been found to be better than a prescriptive diet or vitamin E in the therapy of NAFLD patients receiving nutritional counseling (Am J. Gastroenterol. 2005 May; 100(5):1082-90).

The antihyperlipidemic drug, for example, atorvastatin has been shown to significantly reduce the LDL-cholesterol and liver enzymes in hyperlipidemic patients with biopsy proven NASH. Atorvastatin treatment is reported as effective for NAFLD through a pilot study of atorvastatin treatment in dyslipidemia with nonalcoholic fatty liver patients (Alimentary Pharmacology & Therapeutics. 2006; 23(11):1643-1647).

Hydroxychloroquine has shown antidiabetic effects in some studies. The mechanism by which hydroxychloroquine improves glucose control remains unclear. It appears to lower glucose levels without a large effect on insulin sensitivity or secretion. Data showing that the parent drug chloroquine slows insulin clearance, possibly by stabilizing intracellular lysosomes and slowing the breakdown of the internalized insulin-receptor complex provides one possible explanation for the glucose lowering effect of hydroxychloroquine. This is supported by the fact that hydroxychloroquine inhibits cytosolic insulin metabolism (Diabetes Res Clin Pract. 2002 March; 55(3):209-19).

The glucose lowering efficacy of hydroxychloroquine has been studied through clinical trials. [Diabetes Res Clin Pract. 2002 March; 55(3):209-19; JAMA. 2007 Jul. 11; 298(2):187-93]. Hydroxychloroquine has shown efficacy in patients with resistant diabetes also. A study conducted in 38 patients with non insulin-dependent diabetes resistant to commonly used therapies (oral drugs, insulin, combination of insulin and oral drugs) showed improvements in patients who received insulin and Hydroxychloroquine. The daily insulin dose in patients treated with the combined insulin and hydroxychloroquine therapy had to be reduced by an average of 30% (Ann Intern Med. 1990 May 1; 112(9):678-81).

Hydroxychloroquine has been studied for lipid lowering effects and improvement of serum cholesterol levels in patients treated with hydroxychloroquine has been reported. These include a decrease in serum levels of cholesterol by approximately 10% and an increase in low-density lipoprotein receptors [Ann Rheum Dis. 1997 June; 56(6):374-7; Am J. Med. 1990 September; 89(3):322-6]. Very-low-density lipoprotein cholesterol was reduced in the group receiving hydroxychloroquine, and this was associated with decreased plasma triglycerides in this group (Br J. Rheumatol. 1985 August; 24(3):250-5). Combination treatment of ezetimibe and insulin sensitizing agents are proposed to be more effective than monotherapy in the treatment of NAFLD and among insulin sensitizing agents Rosiglitazone was proposed to be more effective than Metformin in combination therapy (World J Gastroenterol 2006 Jul. 21; 12 (27): 4369-4376).

US20070161578 discloses treatment of non-alcoholic fatty liver disease using cholesterol lowering agents and/or H3 receptor antagonist/inverse agonist.

US20060089412 discloses pharmaceutical composition for the treatment of non-alcoholic fatty liver diseases comprising L-alanine and its combination with Metformin.

Since the cause of NAFLD is multifactorial, and no approved therapy options exist, there is an urgent need in the art to develop effective medications for the treatment of NAFLD, and this becomes the object of the present invention.

### SUMMARY OF THE INVENTION

The present inventor has analyzed that the combination therapy so far known is not so effective to give overall effect on various biochemical parameters indicating cure of NAFLD disease condition and therefore, aims to provide a novel therapeutic combinations that improves the disease condition.

Accordingly, in one aspect, the present invention provides methods for treating non-alcoholic fatty liver disease (NAFLD) as well as methods for prophylaxis of NAFLD or related disorders. The method according to the present invention comprises administering at least three medicaments comprising an amount of hydroxychloroquine or its salts and at least one lipid lowering agent and at least one insulin sensitizing agent to treat subjects suffering from Non-Alcoholic Fatty Liver Disease as well as prophylaxis of Non-Alcoholic Fatty Liver Disease, including humans. In a preferred embodiment, the method comprising administering a combination of hydroxychloroquine and a Statin compound and a biguanide compound, more preferably as a fixed dose combination.

In a further aspect, the present invention provides novel pharmaceutical compositions comprising therapeutically effective amount of hydroxychloroquine or its pharmaceutically acceptable salts; at least one lipid lowering agent and at least one insulin sensitizing agent or a kit containing such combinations.

In a preferred embodiment, the present invention provides synergistic and additive compositions comprising combinations of hydroxychloroquine or its pharmaceutically acceptable salt together with at least one lipid lowering agent and at least one insulin sensitizing agent. The additive and synergistic combinations of the present invention are useful in treating subjects suffering from Non-Alcoholic Fatty Liver Disease and those subjects likely to develop symptoms or signs of Non-Alcoholic Fatty Liver Disease or related disorders.

In another aspect, the invention provides a therapeutic method of lowering/preventing accumulation of fat in liver of a subject which method comprises administering a combination of hydroxychloroquine or its pharmaceutically acceptable salt; a lipid lowering agent and an insulin sensitizing agent in effective amounts.

In yet another aspect, the invention provides a therapeutic method of reducing the liver Index in a subject which method comprises administering a combination of hydroxychloroquine or its pharmaceutically acceptable salt; a lipid lowering agent and an insulin sensitizing agent.

In yet another aspect, the invention provides a therapeutic method of reducing the degree of hepatic injury including steatosis which method comprises administering a combination of hydroxychloroquine or its pharmaceutically acceptable salt; a lipid lowering agent and an insulin sensitizing agent in an effective amounts.

The method according to the above aspects, wherein, the lipid lowering agent is HMG-CoA reductase inhibitor (statins) and insulin sensitizing agent is a biguanide compound. The statin is further selected from lovastatin, atorvastatin, pravastatin, simvastatin, fluvastatin, rosuvastatin or their pharmaceutical salts. The biguanide is metformin or its salt.

In yet further aspect, the invention comprises use of a medicament or a kit comprising fixed dose composition of hydroxychloroquine or its pharmaceutically acceptable salt together with at least one lipid lowering agent and at least one insulin sensitizing agent for treating subjects suffering from Non-Alcoholic Fatty Liver Disease and those subjects likely to develop symptoms or signs of Non-Alcoholic Fatty Liver Disease or related disorders.

The use according to the above aspects, wherein, the lipid lowering agent is HMG-CoA reductase inhibitors (statin) and insulin sensitizing agent is a biguanide compound. The statin is further selected from lovastatin, atorvastatin, pravastatin, simvastatin, fluvastatin, rosuvastatin or their pharmaceutical salts. The biguanide is metformin or its salt.

The invention will now be described in detail in connection with certain preferred and optional embodiments, so that various aspects thereof may be more fully understood and appreciated.

### DESCRIPTION OF DRAWINGS

FIG. 1—Microscopic examination of NAFLD induced liver tissue, serving as control

FIG. 2—Microscopic examination of NAFLD induced liver tissue treated with HCQ

FIG. 3—Microscopic examination of NAFLD induced liver tissue treated with MET

FIG. 4—Microscopic examination of NAFLD induced liver tissue treated with ATV

FIG. 5—Microscopic examination of NAFLD induced liver tissue treated with low dose combination of HCQ and MET

FIG. 6—Microscopic examination of NAFLD induced liver tissue treated with high dose combination of HCQ and MET

FIG. 7—Microscopic examination of NAFLD induced liver tissue treated with low dose combination of HCQ and ATV

FIG. 8—Microscopic examination of NAFLD induced liver tissue treated with high dose combination of HCQ and ATV

FIG. 9—Microscopic examination of NAFLD induced liver tissue treated with low dose combination of HCQ, MET and ATV

FIG. 10—Microscopic examination of NAFLD induced liver tissue treated with high dose combination of HCQ, MET and ATV

### DETAILED DESCRIPTION OF THE INVENTION

Unless specified otherwise, all technical and scientific terms used herein have the same meaning as commonly understood by one of ordinary skill in the art, to which this invention belongs. Although any methods and materials similar or equivalent to those described herein can be used in the practice or testing of the present invention, the preferred methods and materials are described. To describe the invention, certain terms are defined herein specifically as follows:

Unless stated to the contrary, any of the words “including,” “includes,” “comprising,” and “comprises” mean “including without limitation” and shall not be construed to limit any general statement that it follows to the specific or similar items or matters immediately following it. Embodiments of the invention are not mutually exclusive, but may be implemented in various combinations. The described embodiments of the invention and the disclosed examples are given for the purpose of illustration rather than limitation of the invention as set forth the appended claims.

The present invention provides methods for treating non-alcoholic fatty liver disease (NAFLD) as well as methods for prophylaxis of NAFLD or related disorders. The method includes administering novel pharmaceutical compositions comprising therapeutically effective amount of hydroxychloroquine or its pharmaceutically acceptable salts and at least one lipid lowering agent and at least one insulin sensitizing agent. The present compositions may be supplied as a fixed dose combination of said agents or as a kit containing such combinations.

The present invention thus provides synergistic and additive compositions comprising combinations of hydroxychloroquine or its pharmaceutically acceptable salts and at least one lipid lowering agent and at least one insulin sensitizing agent whereby those additive and synergistic combinations are useful in treating subjects suffering from Non-Alcoholic Fatty Liver Disease and those subjects likely to develop symptoms or signs of Non-Alcoholic Fatty Liver Disease or related disorders.

Non-alcoholic fatty liver disease (NAFLD) may include a spectrum of liver disease ranging from simple fatty liver changes (macrovascular fatty change) to non-alcoholic steatohepatitis (NASH), and including cirrhosis that is not related to consumption of alcohol in amounts considered detrimental to the liver or such other symptoms evident of development of liver diseases. The method of treatments also includes prophylaxis measures to prevent development of NAFLD.

The lipid lowering agent according to the invention preferably includes an HMGCoA inhibitor which is selected from statin compounds including lovastatin, atorvastatin, pravastatin, simvastatin, fluvastatin, rosuvastatin etc. or their pharmaceutical salts. More preferably the statin is simvastatin or atorvastatin.

The insulin sensitizing agent according to the invention is antihyperglycemic agents, preferably a biguanide compound. Metformin or its pharmaceutical salt is especially preferred among the biguanide compounds.

Pharmaceutical compositions suitable for use in the present invention include compositions wherein the hydroxychloroquine, statin and biguanide with optional active ingredients are present in an effective amount for treating or preventing NAFLD. According to the invention, the compositions may be prepared as a fixed dose combination of the three drugs, more preferably a fixed dose combination of therapeutically effective amounts of hydroxychloroquine, a statin and a biguanide.

The invention also provides a pharmaceutical kit comprising the above three medicaments, hydroxychloroquine, statin and a biguanide packaged in association with instructions/teaching on method of using the compounds according to one or more of the above-described methods. The kit can contain each of the drug packaged in unit dosage form. The kit may also contain additional pharmaceutically active or inactive agents or compounds.

The term “therapeutically effective amount” means the amount which provides the desired therapeutic effect on administration of the same.

Therapeutic effective amount can be determined by a skilled artisan according to bodyweight of patient, route of administration and condition of disease in a conventional manner. The inventive composition of the present invention for oral administration comprising hydroxychloroquine, for an average adult, in a quantity range from 50 mg to 500 mg. Whereas the biguanide, Metformin for an average adult patient may be used in range of 100 to 2550 mg/day, more preferably in an amount of 250 mg to 2000 mg; whereas the statin may be used in the combination therapy in a quantity range from 2-80 mg, however vary with respect to the specific statin used. In the fixed dose combination of current invention, lovastatin, atorvastatin, simvastatin and rosuvastatin may be used in a quantity range from 2.5 mg to 80 mg respectively, wherein pravastatin may be used in the range of 10-20 mg; lovastatin in the range of 10-20 mg and fluvastatin in the range of 20-80 mg.

The fixed dose compositions according to the invention comprises hydroxychloroquine or its pharmaceutical salt is present in an amount equivalent to 200 mg to 400 mg of free base; Metformin or its salt is present in an amount equivalent to 1000 mg to 2000 mg metformin free base and statin or its pharmaceutical salt in an amount equivalent to 5 to 80 mg drug, however, vary with respect to the specific statin used.

The quantity of the compound/compounds used in fixed dose pharmaceutical compositions of the present invention will vary depending upon the body weight of the patient and the mode of administration and can be of any effective amount to achieve the desired therapeutic effect.

The combination drugs useful in the present invention, or pharmaceutically acceptable salts thereof, can be delivered to the subject using a wide variety of routes or modes of administration. Suitable routes of administration include, but are not limited to, inhalation, transdermal, oral, rectal, transmucosal, intestinal and parenteral administration, including intramuscular, subcutaneous and intravenous injections. A preferred mode of administration includes in oral dosage form. Typically fixed dose combination of hydroxychloroquine, statin and metformin can be administered once or twice a day for long term treatment to a patient suffering with NAFLD. Long term treatment refers to an extended period of time, typically longer than two weeks, and includes any length of time whereby the individual/subject (mammal) exhibits improvement in NAFLD symptoms. This dosage formulation will be beneficial for prophylactic treatment of individuals who will be taking low dosages of the combination for extended periods of time to prevent the development of NAFLD condition.

The fixed dose composition of hydroxychloroquine along with insulin sensitizing agents and statins can be administered in combination with pharmaceutical carriers or diluents. For oral use, suitable pharmaceutical carriers include inert diluents or fillers thereby forming oral dosage forms such as tablets, powders, capsules, syrups or suspensions and the like.

The fixed dose formulation of the present invention is preferably in the form of tablets or capsules, wherein tablets/capsules can be prepared in immediate release, modified/controlled release, extended or in sustain release form. Dosage form may be, for example, but not limited to, a multilayer tablet, a two-layer tablet, or capsules or sachets containing the active ingredients in separate granulates or beads, either granulate or bead, optionally being coated with a protective coating or an enteric-coating.

For example, tablets containing variety of excipients such as disintegrants such as starch, complex silicates together with binding agents such as poly vinyl pyrrolidone, sucrose, gelatin and acacia. Lubricants such as magnesium silicate, sodium lauryl sulfate and talc are often used in tabletting purposes. Solid compositions of the present invention can also be filled into soft and hard gelatin capsules.

For soft gelatin capsule, the composition may be solubilized in suitable vegetable or edible oil such as sunflower oil, corn oil, peanut oil or any other suitable oil.

The method of treatment of administering a combination of Hydroxychloroquine, statin and biguanide, according to the present invention, offers at least one of following advantages:
1. Decreases accumulation of fat in liver
2. Reduces liver Index
3. Reduces increased levels of liver enzymes
4. Reduces the degree of hepatic injury including steatosis

The safety, efficacy and synergy of the fixed dose combination of hydroxychloroquine with a lipid lowering agent (atorvastatin) and insulin sensitizing agent (metformin) in NAFLD is established by the following experiment.

A pharmacological evaluation study performed to compare the efficacy in terms of potency of hydroxychloroquine with or without the insulin sensitizing agent like metformin and lipid lowering agent like atorvastatin. The study was based on the experiment on rats with fatty liver induced by fructose feeding and given oral doses of different strengths of the corresponding drug samples for 14 days. It was concluded from the study that the protection given by the combination therapy is more as compared to monotherapy.

Pharmacological Evaluation of Combination of Hydroxychloroquine with Atorvastatin and Metformin

An experimental study was conducted in Institute of Pharmaceutical Research and Education, Wardha, India to demonstrate the efficacy of fixed dose combination of hydroxychloroquine (HCQ) with atorvastatin (ATV) and metformin (MET) in NAFLD. In this experiment 66 Wistar rats weighing 150-200 g of either sex were divided randomly into 11 groups consisting of six rats each. Group 1 (normal control) consisted of normal rats that neither received fructose nor any drug, Group 2 served as NAFLD induced control (NAFLD was induced in rats by replacing water with 10% fructose solution for two weeks) which received vehicle orally, Group 3 was NAFLD induced and treated with HCQ (160 mg/kg), Group 4 was NAFLD induced and treated with ATV (8 mg/kg), Group 5 was NAFLD induced and treated with MET (500 mg/kg), Group 6 was NAFLD induced and treated with low dose combination of HCQ (80 mg/kg) and MET (250 mg/kg). Group 7 was NAFLD induced and treated with high dose combination of HCQ (160 mg/kg) and MET (500 mg/kg). Group 8 was NAFLD induced and treated with low dose combination of HCQ (80 mg/kg) and ATV (4 mg/kg). Group 9 was NAFLD induced and treated with high dose combination of HCQ (160 mg/kg) and ATV (8 mg/kg). Group 10 was NAFLD induced and treated with low dose combination of ATV (4 mg/kg), HCQ (80 mg/kg) and MET (250 mg/kg) whereas Group 11 was NAFLD induced and treated with high dose combination of ATV (8 mg/kg), HCQ (160 mg/kg) and MET (500 mg/kg). The study drugs suspended in vehicle [0.1% w/v suspension of Tween 80 and carboxymethylcellulose (CMC) in water] were administered for 14 days. After 14 days treatment, all the animals were sacrificed under chloroform anesthesia and blood was collected for determination of blood glucose levels, triglyceride levels, SGPT, SGOT, alkaline phosphatase and total cholesterol using autoanalyser (Microlab 2000) Liver index was also calculated based upon the body weight and liver weight. At the end of the therapy histopathological evaluations were performed.

At the start of the experiment, baseline parameters did not differ significantly between the groups. As compared to normal group, the serum levels of total cholesterol, triglycerides, alkaline phosphatase, SGOT, SGPT and glucose in NAFLD control group increased significantly. After 14 days, in combination treated group, reduction in serum total cholesterol, triglyceride and level of liver enzymes like alkaline phosphatase, serum glutamate oxaloacetate transaminase (SGOT), serum glutamic pyruvic transaminase (SGPT) was comparable to ATV monotherapy treated group. The reduction in glucose levels with combination therapy was comparable to MET monotherapy, but the lipid levels and liver enzyme levels were significantly reduced in combination treated group as compared to MET monotherapy.

It was observed that the liver index calculated based on the body weight and liver weight of NAFLD control rats was higher (4.50±0.04) than the normal rats (2.71±0.22). After 14 days of administration, combination of HCQ, MET and ATV reduced the liver index significantly (P<0.001) as compared to groups treated with HCQ, ATV and MET alone. Moreover, the high dose combination of HCQ+ATV+MET treated rats showed maximum reduction as compared to fatty liver rats.

Effects of HCQ, ATV and MET on various biochemical parameters and on liver in fructose induced fatty liver is detailed below in Table I

TABLE 1
Effects of HCQ and ATV on various biochemical parameters and on liver in fructose induced fatty liver

| Sr. | | Weight (g) | Weight (g) | Glucose | TC | TG |
|---|---|---|---|---|---|---|
| No | Groups | Initial | Final | (mg/dl) | (mg/dl) | (mg/dl) |
| 1. | Normal | 166.66 ± 6.59 | 182 ± 6.63 | 126.16 ± 8.79 | 128.66 ± 4.84 | 443.66 ± 40.90 |
| 2. | Control | 160.16 ± 5.38 | 181.66 ± 3.32 | 159.00 ± 10.65** | 161.33 ± 10.93** | 583 ± 38.82** |
| | (NAFLD) | | | | | |
| 3. | HCQ | 158 ± 7.18 | 168.5 ± 5.35 | 157.16 ± 7.93 | 159.66 ± 10.23 | 556.66 ± 12.21* |
| 4. | ATV | 157.5 ± 6.02 | 171.66 ± 6.77 | 159.33 ± 9.07 | 137.66 ± 5.20** | 463.33 ± 12.12** |
| 5. | MET | 157.33 ± 5.46 | 169.83 ± 8.63 | 125.16 ± 6.24 | 155.66 ± 10.85 | 516.33 ± 56.25 |
| 6. | HCQ + MET | 159.16 ± 4.07 | 171.33 ± 6.47 | 142.33 ± 6.28 | 158.83 ± 5.63 | 524.5 ± 11.67 |
| | (Low dose) | | | | | |
| 7. | HCQ + MET | 160.66 ± 6.97 | 173.5 ± 5.61 | 118.33 ± 9.13 | 147.33 ± 7.17 | 502.5 ± 10.42 |
| | (High dose) | | | | | |
| 8. | HCQ + ATV | 158.66 ± 6.21 | 177.66 ± 9.09 | 160.16 ± 8.18 | 144.16 ± 9.86** | 477.00 ± 47.50** |
| | (Low Dose) | | | | | |
| 9. | HCQ + ATV | 169.83 ± 10.90 | 187.66 8.75 | 158.16 7.62 | 135.33 ± 7.00** | 466.83 ± 30.45** |
| | (High Dose) | | | | | |
| 10. | HCQ + ATV + | 158.666 ± 6.25 | 172.66 ± 5.81 | 129.5 ± 10.03** | 144.83 ± 5.98* | 472.16 ± 6.73** |
| | MET (Low | | | | | |
| | dose) | | | | | |
| 11. | HCQ + ATV + | 157.16 ± 5.23 | 175.83 ± 3.65* | 124 ± 7.04** | 134.16 ± 4.91** | 451.66 ± 13.99** |
| | MET (High | | | | | |
| | doae) | | | | | |

| Sr. | Alk. PO₄ | SGPT | SGOT | Liver | Liver |
|---|---|---|---|---|---|
| No | (U/L) | (U/L) | (U/L) | Index | Weight (g) |
| 1. | 49.00 ± 1.41 | 30.00 ± 1.41 | 96 ± 3.89 | 2.72 ± 0.22 | 4.96 ± 0.58 |
| 2. | 67.83 ± 2.48** | 51 ± 5.25** | 145.33 ± 7.28** | 4.50 ± 0.04** | 8.18** ± 0.22 |
| 3. | 63.16 ± 4.07* | 44.83 ± 2.78 | 137.83 ± 10.59 | 4.53 ± 0.10 | 7.64 ± 0.33 |
| 4. | 54.66 ± 2.58** | 37.5 ± 2.42** | 114.00 ± 3.50** | 3.20 ± 0.24** | 5.51** ± 0.52 |
| 5. | 61 ± 6.32 | 43.66 ± 7.33 | 132.33 ± 8.98 | 4.50 ± 0.30 | 7.63 ± 0.30 |
| 6. | 63.5 ± 3.27 | 48.5 ± 3.01 | 143.5 ± 3.50 | 4.27 ± 0.26 | 7.31 ± 0.38 |
| 7. | 59.66 ± 3.93 | 43 ± 4.81 | 134.9.81 | 4.26 ± 00.17 | 7.39 ± 0.38 |
| 8. | 59.50** ± 7.36 | 41.33 ± 7.96** | 135.66 ± 7.36 | 2.95 ± 0.19** | 5.23** ± 0.20 |
| 9. | 52.16 ± 2.63** | 36.00 ± 3.22** | 106.33 ± 4.17** | 3.05 ± 0.38** | 5.70 ± 0.58** |
| 10. | 56.5 ± 4.18** | 40.16 ± 4.87* | 117.33 ± 7.22** | 3.94 ± 0.16** | 6.80 ± 0.30** |
| 11. | 51.33 ± 3.77** | 35.66 ± 2.16** | 105.83 ± 3.86** | 3.08 ± 0.26** | 5.42 ± 0.44** |

Values are expressed as Mean ± S.D. (n = 6),
*p < 0.05 and
**p < 0.01.
HCQ—Hydroxychloroquine, MET—Metformin and ATV—Atorvastatin.
TC: Total Cholesterol
TG: Triglyceride

### Results of Histopathological Examination

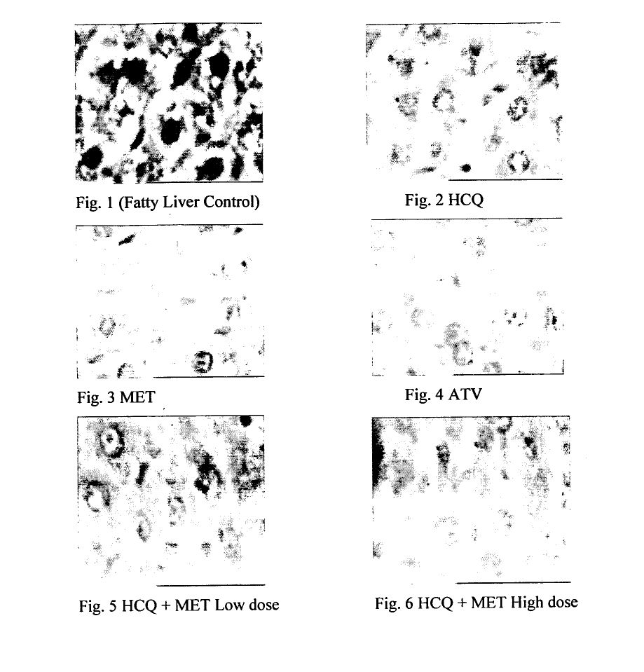

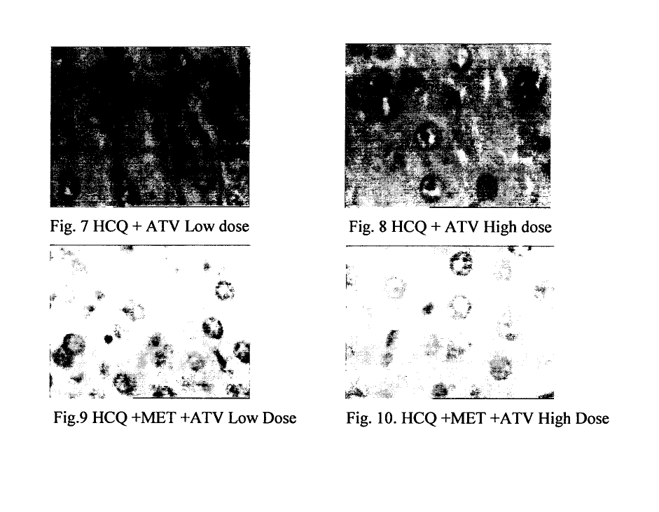

Histopathological examination after 14 days administration of 10% fructose in vehicle control group showed typical steatosis accompanied by multiple vacuoles and fatty infiltration (FIG. 1). Treatment with HCQ (160 mg/kg p.o.) alone did not show any recovery from fatty change (FIG. 2). However, the degree of hepatic injury including steatosis and fatty infiltration were attenuated in ATV (8 mg/kg p.o.) treated group (FIG. 4). Liver cells showed regeneration and repair in the form of nucleolation and absence of coarse fat vacuoles in group treated with Metformin(FIG. 3). Liver showed normal hepatic parenchyma with repair changes when treated with combination of HCQ+MET Low dose and HCQ+MET High dose respectively (FIGS. 5 & 6). Similarly, Liver showed normal hepatic parenchyma with repair changes when treated with HCQ+ATV Low dose (FIG. 7) and HCQ+ATV High dose (FIG. 8). In group treated with combination of HCQ, ATV and MET, changes of recovery were more as compared to groups treated with monotherapy (FIGS. 9&10).

After 14 days administration of low and high dose combination of ATV, HCQ and MET reduced the liver index significantly as compared to monotherapy (FIGS. 9 & 10) Moreover high dose combination of ATV 10 mg/kg+HCQ 200 mg/kg+MET 500 mg/kg (FIG. 10) treated rats showed maximum reduction as compared to fatty liver rats.

Regenerating hepatocytes were with extravacation of blood intracytoplasmic bile plugs can be seen from FIGS. 9 to 10.

The results of pharmacological evaluation reveals that the current invention shows for the first time that the combination therapy of hydroxychloroquine with atorvastatin and metformin has a greater effect on hypertriglyceridemia and hepatic injury including steatosis and fatty infiltration compared to insulin sensitizing agent, lipid lowering agent monotherapy.

The following examples, which include preferred embodiments, will serve to illustrate the practice of this invention, it being understood that the particulars shown are by way of example and for purpose of illustrative discussion of preferred embodiments of the invention.

### Example 1

### i. Each Tablet/Capsule Contains

| | |
|---|---|
| Hydroxychloroquine | 200 mg to 400 mg |
| Atorvastatin | 5 mg to 40 mg |
| Metformin (conventional or modified release | 1000 mg to 2000 mg |
| or sustained release or controlled release) | |

### Example 2

### i. Each Tablet/Capsule Contains

| | |
|---|---|
| Hydroxychloroquine | 200 mg to 400 mg |
| Simvastatin | 5 mg to 40 mg |
| Metformin (conventional or modified release | 1000 mg to 2000 mg |
| or sustained release or controlled release) | |

### Example 3

### i. Each Tablet/Capsule Contains

| | |
|---|---|
| Hydroxychloroquine | 200 mg to 400 mg |
| Pravastatin | 10 mg to 20 mg |
| Metformin (conventional or modified release | 1000 mg to 2000 mg |
| or sustained release or controlled release) | |

### Example 4

### i. Each Tablet/Capsule Contains

| | |
|---|---|
| Hydroxychloroquine | 200 mg to 400 mg |
| Lovastatin | 10 mg to 20 mg |
| Metformin (conventional or modified release | 1000 mg to 2000 mg |
| or sustained release or controlled release) | |

### Example 5

### i. Each Tablet/Capsule Contains

| | |
|---|---|
| Hydroxychloroquine | 200 mg to 400 mg |
| Rosuvastatin | 5 mg to 40 mg |
| Metformin (conventional or modified release | 1000 mg to 2000 mg |
| or sustained release or controlled release) | |

### Example 6

### i. Each Tablet/Capsule Contains

| | |
|---|---|
| Hydroxychloroquine | 200 mg to 400 mg |
| Fluvastatin | 20 mg to 80 mg |
| Metformin (conventional or modified release | 1000 mg to 2000 mg |
| or sustained release or controlled release) | |

It will be evident to those skilled in the art that the invention is not limited to the details of the foregoing illustrative examples and that the present invention may be embodied in other specific forms without departing from the essential attributes thereof, and it is therefore desired that the present embodiments and examples be considered in all respects as illustrative and not restrictive, reference being made to the appended claims, rather than to the foregoing description, and all changes which come within the meaning and range of equivalency of the claims are therefore intended to be embraced therein.

1. A synergistic pharmaceutical combination comprising:
a. hydroxychloroquine or its pharmaceutically acceptable salt;
b. one or more lipid lowering agent; and
c. one or more an insulin sensitizing agent.

2. (canceled)

3. The pharmaceutical combination according to claim 1, wherein said lipid lowering agent is HMG-CoA reductase inhibitor.

4. The pharmaceutical combination according to claim 3, wherein said HMG-CoA reductase inhibitor is selected from the group consisting of atorvastatin, pravastatin, lovastatin, simvastatin, fluvastatin, rosuvastatin, and their pharmaceutical salts.

5. The pharmaceutical combination according to claim 1, wherein said insulin sensitizing agent is a biguanide compound.

6. The pharmaceutical combination according to claim 5, wherein said biguanide is metformin or its pharmaceutical salts.

7. The pharmaceutical combination according to claims 1, wherein the lipid lowering agent is a HMG-CoA reductase inhibitor, and the insulin sensitising agent is metformin or a pharmaceutical salt thereof.

8. The pharmaceutical combination according to claim 7, wherein hydroxychloroquine or its pharmaceutically acceptable salt is present in an amount equivalent to 200 mg to 400 mg of free base; metformin or its salt is present in an amount equivalent to 1000 mg to 2000 mg metformin base, and the HMG-CoA reductase inhibitor is present in an amount equivalent to 5 to 80 mg drug.

9-10. (canceled)

11. A method for treating or preventing non-alcoholic fatty liver disease (NAFLD) or related liver disorders in a subject, lowering accumulation of fat in a liver or reducing/normalising the fatty liver index of a subject, or reducing the degree of hepatic injury in a subject, the method comprising administering to said subject a combination of hydroxychloroquine or its pharmaceutically acceptable salt, and at least one lipid lowering agent or at least one insulin sensitizing agent.

12-13. (canceled)

14. The method according to claim 11, wherein the lipid lowering agent is HMG-CoA reductase inhibitor.

15. The method according to claim 14, wherein said HMG-CoA reductase inhibitor is selected from the group consisting of atorvastatin, pravastatin, lovastatin, simvastatin, fluvastatin, rosuvastatin, and their pharmaceutical salts.

16. The method according to claim 22, wherein said biguanide compound is metformin or its pharmaceutical salt.

17-21. (canceled)

22. The method according to claim 11, wherein the insulin sensitizing agent is a biguanide compound.

23. The method according to claim 16, wherein metformin or its salt is present in an amount equivalent to 1000 mg to 2000 mg metformin base.

24. The method according to claim 14, wherein the HMG-CoA reductase inhibitor is present in an amount equivalent to 5 to 80 mg drug.

25. The method according to claim 11, wherein hydroxychloroquine or its pharmaceutically acceptable salt is present in an amount equivalent to 200 mg to 400 mg of free base.

26. The method according to claim 11, wherein said combination is formulated as fixed dose combination or a pharmaceutical kit containing unit dosage forms for sequential/simultaneous administration.

27. The method according to claim 11, wherein the lipid lowering agent is a statin and the insulin sensitizing agent is a biguanide compound.

28. The method according to claim 27, wherein said statin is selected from the group consisting of atorvastatin, pravastatin, lovastatin, simvastatin, fluvastatin, rosuvastatin, and their pharmaceutically acceptable salts.

29. The method according to claim 27, wherein said biguanide is metformin or a pharmaceutically acceptable salt thereof.

30. The method according to claim 11, wherein the combination contains hydroxychloroquine or its pharmaceutically acceptable salt, and atorvastatin or metformin.
